SEQUENCE LISTING (62 sequences)

SEQ ID NO: 1 - protein, 221 aa, Vibrio cholerae
MGYVRVIYQFPDNLWWNECTNQVYYAQDPMKPERLIGTPSIIQTKLLKILCEYHPAPCPNDQIIKALWPHGFISSESLTQAIKRTRDFLNDEHKTLIENVKLQGYRINIIQVIVSENVVDEADCSQKKSVKERIKIEWGKINVVPYLVFSALYVALLPVIWWSYGQWYQHELAGITHDLRDLARLPGITIQKLSEQKLTFAIDQHQCSVNYEQKTLECTKN

SEQ ID NO: 2 - protein, 136 aa, Vibrio cholerae
MHKKLKAWGGATGLFVVALGVTIIALPMRQKNSHGTMIIDGTVTQIFSTYQGNLSNVWLTQTDPQGNVVKSWTTRYQTLPDPSSQKLNLIPDYSQSNASRDYNVLSIYQLGKGCFLAFPYKLLTAEKMWFSCQSDF

SEQ ID NO: 3 - protein, 58 aa, Artificial
  Synthetic bile salts sensing domain
  MISC_FEATURE (47)..(47): X represents N, D, W, Y, T, V or F
  MISC_FEATURE (48)..(48): X represents Y or F
  MISC_FEATURE (49)..(49): X represents E, G, V, I, L, S or K
  MISC_FEATURE (50)..(50): X represents Q, V, H, A, T, D, L or S
YGQWYQHELAGITHDLRDLARLPGITIQKLSEQKLTFAIDQHQCSVXXXXKTLECTKN

SEQ ID NO: 4 - protein, 4 aa, Artificial
  Synthetic variant of the bile salts sensing domain
NYEQ

SEQ ID NO: 5 - protein, 4 aa, Artificial
  Synthetic variant of the bile salts sensing domain
DFGV

SEQ ID NO: 6 - protein, 4 aa, Artificial
  Synthetic variant of the bile salts sensing domain
WYVH

SEQ ID NO: 7 - protein, 4 aa, Artificial
  Synthetic variant of the bile salts sensing domain
YYIV

SEQ ID NO: 8 - protein, 4 aa, Artificial
  Synthetic variant of the bile salts sensing domain
TFLA

SEQ ID NO: 9 - protein, 4 aa, Artificial
  Synthetic variant of the bile salts sensing domain
DFLT

SEQ ID NO: 10 - protein, 4 aa, Artificial
  Synthetic variant of the bile salts sensing domain
VFSD

SEQ ID NO: 11 - protein, 4 aa, Artificial
  Synthetic variant of the bile salts sensing domain
FFKA

SEQ ID NO: 12 - protein, 4 aa, Artificial
  Synthetic variant of the bile salts sensing domain
YYVL

SEQ ID NO: 13 - protein, 4 aa, Artificial
  Synthetic variant of the bile salts sensing domain
FYES

SEQ ID NO: 14 - DNA, 12 bp, Artificial
  Synthetic nucleic variant of the bile salts sensing domain
aattacgaacaa

SEQ ID NO: 15 - DNA, 12 bp, Artificial
  Synthetic nucleic variant of the bile salts sensing domain
gattttggggtg

SEQ ID NO: 16 - DNA, 12 bp, Artificial
  Synthetic nucleic variant of the bile salts sensing domain
tggtatgttcat

SEQ ID NO: 17 - DNA, 12 bp, Artificial
  Synthetic nucleic variant of the bile salts sensing domain
tattatattgtt

SEQ ID NO: 18 - DNA, 12 bp, Artificial
  Synthetic nucleic variant of the bile salts sensing domain
acttttcttgct

SEQ ID NO: 19 - DNA, 12 bp, Artificial
  Synthetic nucleic variant of the bile salt sensing domain
gattttcttact

SEQ ID NO: 20 - DNA, 12 bp, Artificial
  Synthetic nucleic variant of the bile salts sensing domain
gttttttcggat

SEQ ID NO: 21 - DNA, 12 bp, Artificial
  Synthetic nucleic variant of the bile salts sensing domain
ttttttaaggcg

SEQ ID NO: 22 - DNA, 12 bp, Artificial
  Synthetic nucleic variant of the bile salts sensing domain
tattatgttctt

SEQ ID NO: 23 - DNA, 12 bp, Artificial
  Synthetic nucleic variant of the bile salts sensing domain
ttttatgagagt

SEQ ID NO: 24 - protein, 58 aa, Artificial
  Synthetic bile salts sensing domain
YGQWYQHELAGITHDLRDLARLPGITIQKLSEQKLTFAIDQHQCSVNYEQKTLECTKN

SEQ ID NO: 25 - protein, 58 aa, Artificial
  Synthetic bile salts sensing domain
YGQWYQHELAGITHDLRDLARLPGITIQKLSEQKLTFAIDQHQCSVDFGVKTLECTKN

SEQ ID NO: 26 - protein, 58 aa, Artificial
  Synthetic bile salts sensing domain
YGQWYQHELAGITHDLRDLARLPGITIQKLSEQKLTFAIDQHQCSVWYVHKTLECTKN

SEQ ID NO: 27 - protein, 58 aa, Artificial
  Synthetic bile salts sensing domain
YGQWYQHELAGITHDLRDLARLPGITIQKLSEQKLTFAIDQHQCSVYYIVKTLECTKN

SEQ ID NO: 28 - protein, 58 aa, Artificial
  Synthetic bile salts sensing domain
YGQWYQHELAGITHDLRDLARLPGITIQKLSEQKLTFAIDQHQCSVTFLAKTLECTKN

SEQ ID NO: 29 - protein, 58 aa, Artificial
  Synthetic bile salts sensing domain
YGQWYQHELAGITHDLRDLARLPGITIQKLSEQKLTFAIDQHQCSVDFLTKTLECTKN

SEQ ID NO: 30 - protein, 58 aa, Artificial
  Synthetic bile salts sensing domain
YGQWYQHELAGITHDLRDLARLPGITIQKLSEQKLTFAIDQHQCSVVFSDKTLECTKN

SEQ ID NO: 31 - protein, 58 aa, Artificial
  Synthetic bile salts sensing domain
YGQWYQHELAGITHDLRDLARLPGITIQKLSEQKLTFAIDQHQCSVFFKAKTLECTKN

SEQ ID NO: 32 - protein, 58 aa, Artificial
  Synthetic bile salts sensing domain
YGQWYQHELAGITHDLRDLARLPGITIQKLSEQKLTFAIDQHQCSVYYVLKTLECTKN

SEQ ID NO: 33 - protein, 58 aa, Artificial
  Synthetic bile salts sensing domain
YGQWYQHELAGITHDLRDLARLPGITIQKLSEQKLTFAIDQHQCSVFYESKTLECTKN

SEQ ID NO: 34 - protein, 79 aa, Artificial
  Synthetic TcpP polypeptide
  MISC_FEATURE (68)..(68): X represents N, D, W, Y, T, V or F
  MISC_FEATURE (69)..(69): X represents represents Y or F
  MISC_FEATURE (70)..(70): X represents E, G, V, I, L, S or K
  MISC_FEATURE (71)..(71): X represents Q, V, H, A, T, D, L or S
VVPYLVFSALYVALLPVIWWSYGQWYQHELAGITHDLRDLARLPGITIQKLSEQKLTFAIDQHQCSVXXXXKTLECTKN

SEQ ID NO: 35 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVNYEQKTLECTKN

SEQ ID NO: 36 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVDFGVKTLECTKN

SEQ ID NO: 37 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVWYVHKTLECTKN

SEQ ID NO: 38 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVYYIVKTLECTKN

SEQ ID NO: 39 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVTFLAKTLECTKN

SEQ ID NO: 40 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVDFLTKTLECTKN

SEQ ID NO: 41 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVVFSDKTLECTKN

SEQ ID NO: 42 - protein, 78 aa, Artificial
  Synthetic TcpP polypeptide
VVPYLVFSALYVALLPVIWWSYGQWYQHELAGITHDLRDLARLPGITIQKLSEQKLTFAIDQHQCSVFFKAKTLECTK

SEQ ID NO: 43 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVYYVLKTLECTKN

SEQ ID NO: 44 - protein, 79 aa, Artificial
  Synthetic TcpP polypeptide
VVPYLVFSALYVALLPVIWWSYGQWYQHELAGITHDLRDLARLPGITIQKLSEQKLTFAIDQHQCSVFYESKTLECTKN

SEQ ID NO: 45 - protein, 107 aa, Artificial
  Synthetic E coli CadC transcriptional activator DNA binding domain
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWY

SEQ ID NO: 46 - protein, 48 aa, Artificial
  Synthetic linker
SEEEGEEIMLSSPPPIPEAVPATDSPSHSLNIQNTATPPEQSPVKSKR

SEQ ID NO: 47 - protein, 234 aa, Artificial
  Synthetic bile salts chimeric receptor
  MISC_FEATURE (223)..(223): X represents N, D, W, Y, T, V or F
  MISC_FEATURE (224)..(224): X represents Y or F
  MISC_FEATURE (225)..(225): X represents E, G, V, I, L, S or K
  MISC_FEATURE (226)..(226): X represents Q, V, H, A, T, D, L or S
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVXXXXKTLECTKN

SEQ ID NO: 48 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVNYEQKTLECTKN

SEQ ID NO: 49 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVDFGVKTLECTKN

SEQ ID NO: 50 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVWYVHKTLECTKN

SEQ ID NO: 51 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVYYIVKTLECTKN

SEQ ID NO: 52 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVTFLAKTLECTKN

SEQ ID NO: 53 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVDFLTKTLECTKN

SEQ ID NO: 54 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVVFSDKTLECTKN

SEQ ID NO: 55 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVFFKAKTLECTKN

SEQ ID NO: 56 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVYYVLKTLECTKN

SEQ ID NO: 57 - protein, 234 aa, Artificial
  Synthetic bile salts chimeric receptor
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRVVPYLVFSALYVALLPVIWWSYGQWYQHELAGITHDLRDLARLPGITIQKLSEQKLTFAIDQHQCSVFYESKTLECTKN

SEQ ID NO: 58 - DNA, 36 bp, Artificial
  Synthetic p14 promoter
ttgacaattaatcatccggctcgtataatgtgtgga

SEQ ID NO: 59 - DNA, 36 bp, Artificial
  Synthetic p10 promoter
tttcaatttaatcatccggctcgtataatgtgtgga

SEQ ID NO: 60 - DNA, 35 bp, Artificial
  Synthetic p9 promoter
ttgcctcttaatcatcggctcgtataatgtgtgga

SEQ ID NO: 61 - DNA, 36 bp, Artificial
  Synthetic p5 promoter
ttgacaattaatcatccggctcgtaatttatgtgga

SEQ ID NO: 62 - DNA, 162 bp, Artificial
  Synthetic PCadBA promoter
atccattgtaaacattaaatgtttatcttttcatgatatcaacttgcgatcctgatgtgttaataaaaaacctcaagttctcacttacagaaacttttgtgttatttcacctaatctttaggattaatccttttttcgtgagtaatcttatcgccagtttgg

(19) United States
(12) Patent Application Publication
(10) Pub. No.: US 2023/0304023 A1
(43) Pub. Date: Sep. 28, 2023

(54) BILE SALTS BACTOSENSOR AND USE THEREOF FOR DIAGNOSTIC AND THERAPEUTIC PURPOSES

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ DE MONTPELLIER, Montpellier (FR)

(72) Inventors: Jérôme BONNET, MONTPELLIER (FR); Hung Ju CHANG, MONTPELLIER (FR); Jérôme GRACY, MONTPELLIER (FR); Martin COHEN-GONSAUD, MONTPELLIER (FR)

(21) Appl. No.: 18/002,215

(22) PCT Filed: Jun. 16, 2021
(86) PCT No.: PCT/EP2021/066224

§ 371 Date: Dec. 16, 2022

(30) Foreign Application Priority Data
Jun. 17, 2020 (EP) 20305662.7

(51) Int. Cl. C12N 15/63; C07K 14/28; C12N 15/70

(52) U.S. Cl. CPC C12N 15/635; C07K 14/28; C12N 15/70; C07K 2319/71

(57) ABSTRACT

Bile salts are steroid acids derived from cholesterol in the liver, are released into the gastrointestinal tract to aid in digestion and are thoroughly modified by the resident gut microbiota. Bile acids act as versatile signaling molecules with a variety of endocrine functions and are linked to several diseases. In particular, serum and urinary bile salts represent biomarkers for early diagnostics of liver dysfunction, yet their current detection methods are impractical and hard to scale. Here the inventors engineered engineered synthetic bile salt receptors using TcpP as sensing domains connected to E. coli CadC system which activates transcription upon dimerization. The performance of the system was assayed for various selection of promoters and they can show that fine tunable response that may be reached by changing expression levels of the bile salt receptor. By performing multiple rounds of directed evolution of the TcpP sensor the inventors obtained a collection of variants with a lower limit of detection and a higher sensitivity. Finally, they show that their bactosensor can detect pathological bile-salt concentrations in samples from patients with liver dysfunction. The present invention thus relates to bile salts bactosensor and use thereof for diagnostic and therapeutic purposes.

### FIELD OF THE INVENTION

The present invention is in the field of medicine, in particular synthetic biology and hepatology.

### BACKGROUND OF THE INVENTION

The liver is a vital organ coordinating metabolic, detoxification, and immunological processes. Liver diseases including hepatitis, cirrhosis, fatty liver disease and cancer are major public health problems and require large-scale screening methods for prevention, diagnosis, and therapeutic monitoring. Liver biopsy and ultrasound-based elastography are the most common methods for the diagnosis and monitoring the progress of liver diseases. However, these technologies are still limited by the requirement of sophisticated infrastructures and well-trained technicians. Liver function can also be monitored by quantifying serum enzymatic activities and bilirubin, but these markers are detectable when damage has already progressed, and are not entirely specific. Liver function is usually monitored by quantifying several enzymatic activities simultaneously due to their lack of specificity. Serum and urinary bile salts are alternative biomarkers for early diagnostics of liver dysfunction, yet their current detection methods are impractical and hard to scale.

WO2018049362 discloses bile salts sensors, in particular to transcriptional sensor for bile salts in Bacteroides thetaiotaomicron, using bile sensor proteins such as BreR and VFA0359 from Vibrio fischeri.

### SUMMARY OF THE INVENTION

The present invention is defined by the claims. More particularly, the present invention relates to bile salts bactosensor and use thereof for diagnostic and therapeutic purposes.

### DETAILED DESCRIPTION OF THE INVENTION

### Definitions

As used herein, the term “amino acid residue” is intended to include any natural or synthetic amino acid residue, and is primarily intended to indicate an amino acid residue contained in the group consisting of the 20 naturally occurring amino acids, i.e. selected from the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. According to the invention a first amino acid sequence having at least 90% of identity with a second amino acid sequence means that the first sequence has 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. ApplsBiosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

As used herein, the expression “derived from” refers to a process whereby a first component (e.g., a first polypeptide), or information from that first component, is used to isolate, derive or make a different second component (e.g., a second polypeptide that is different from the first one).

As used herein, the term “fusion protein” refers to a single polypeptide chain having at least two polypeptide domains that are not normally present in a single, natural polypeptide. Thus, naturally occurring proteins are not “fusion proteins”, as used herein. Preferably, a polypeptide of interest (e.g. TcpP polypeptide) is fused with at least one heterologous polypeptide (e.g. DNA binding domain) via a peptide bond and the fusion protein may also include the linking regions of amino acids between amino acid portions derived from separate proteins.

As used herein, the term “heterologous polypeptide” refers to a polypeptide which does not derive from the same protein to which said heterologous polypeptide is fused.

As used herein, the term “linker” refers to a sequence of at least one amino acid that links the polypeptide of interest to the heterologous polypeptide in the fusion protein. Such a linker may be useful to prevent steric hindrances. Typically a linker comprises 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; or 70 amino acids.

As used herein, the term “TcpP” refers to the toxin coregulated pilus biosynthesis protein P of Vibrio cholerae. TcpP is a transmembrane transcription factor and it has been shown that a set of bile salts cause dimerization of the transmembrane transcription factor TcpP by inducing intermolecular disulfide bonds in its periplasmic domain. In particular, the TcpP protein comprises a transmembrane domain and a periplasmic sensing domain. An exemplary amino acid sequence of TcpP is shown as SEQ ID NO:1.

SEQ ID NO:1 >sp | P29485 | TCPP_VIBCH Toxin coregulated pilus biosynthesis protein P OS=Vibrio cholerae serotype 01 (strain ATCC 39315 / El Tor Inaba N16961) OX=243277 GN=tcpP PE=4 SV=2; the transmembrane domain is indicated in bold, italic and underlined. The periplasmic sensing domain is indicated in bold and double underlined.

MGYVRVIYQFPDNLWWNECTNQVYYAQDPMKPERLIGTPSIIQTKLLKIL
CEYHPAPCPNDQIIKALWPHGFISSESLTQAIKRTRDFLNDEHKTLIENV
KLQGYRINIIQVIVSENVVDEADCSQKKSVKERIKIEWGKINVVPYLVFS ALYVALLPVIWWSYGQHELAGITHDLRDLARLPGITIQKLSEQKLTFAID QHQCSVNYEQKTLECTKN

As used herein, the term “TcpH” refers to the toxin coregulated pilus biosynthesis protein H of Vibrio cholerae. An exemplary amino acid sequence of TcpH is shown as SEQ ID NO:2.

SEQ ID NO:2 >sp | P29489 | TCPH_ VIBCH Toxin coreg
ulated pilus biosynthesisprotein H OS=Vibrio chole
rae serotype 01 (strain ATCC 39315 / El Tor InabaN
16961) OX=243277 GN=TcpH PE=4 SV=2MHKKLKAWGGATGLFV
VALGVTIIALPMRQKNSHGTMIIDGTVTQIFSTYQGNLSNVWLTQTDPQG
NVVKSWTTRYQTLPDPSSQKLNLIPDYSQSNASRDYNVLSIYQLGKGCFL
AFPYKLLTAEKMWFSCQSDF

As used herein, the term “DNA binding domain” refers to, but is not limited to, a motif that can bind to a specific DNA sequence (e.g., a genomic DNA sequence). DNA binding domains have at least one motif that recognizes and binds to single-stranded or double-stranded DNA. DNA binding domains can interact with DNA in a sequence-specific or a non-sequence-specific manner.

As used herein, the term “CadC transcriptional activator” has its general meaning in the art and refers to the membrane-integrated transcriptional regulator CadC of Escherichia coli. CadC activates expression of the cadBA operon at low external pH with concomitantly available lysine, providing adaptation to mild acidic stress. CadC is a representative of the ToxR-like proteins that combine sensory, signal transduction, and DNA-binding activities within a single polypeptide. Specifically, CadC is composed of a C-terminal periplasmic pH-sensing domain, a single transmembrane helix and an N-terminal cytoplasmic winged helix-turn-helix DNA-binding domain (Buchner S, Schlundt A, Lassak J, Sattler M, Jung K. Structural and Functional Analysis of the Signal-Transducing Linker in the pH-Responsive One-Component System CadC of Escherichia coli. J Mol Biol. 2015 Jul 31;427(15):2548-61.). CadC dimerizes via its C-terminal periplasmic pH-sensing domain. Thus the expression “ E coli CadC transcriptional activator DNA binding domain” refers to the cytoplasmic domain of CadC that is capable of restoring its function via oligomerization of its C-terminal fusion domain.

As used herein, the term “recombinant” refers to an artificial combination of two otherwise separated segments of sequence, e.g., by chemical synthesis or by the manipulation of isolated segments of amino acids or of nucleic acids by genetic engineering techniques.

As used herein, the term “expression cassette” refers to a nucleic acid sequence that is capable in an appropriate setting of driving the expression of a polynucleotide encoding a polypeptide of interest that is incorporated in said expression cassette. When introduced into a host cell, an expression cassette inter alia is capable of directing the cell’s machinery to transcribe an incorporated polynucleotide encoding a polypeptide of interest into RNA, which is then usually further processed and finally translated into the polypeptide of interest. The expression cassette can be comprised in an expression vector as will be described in further detail below. The individual elements of the expression cassette according to the present invention are subsequently explained in detail.

As used herein, the term “promoter” refers to a nucleic acid sequence that facilitates the transcription of a polynucleotide of interest. The promoter is operably linked to the polynucleotide of interest. The promoter may also form part of a promoter/enhancer element. Although the physical boundaries between the elements “promoter” and “enhancer” are not always clear, the term “promoter” usually refers to a site on the nucleic acid molecule to which an RNA polymerase and/or any associated factors binds and at which transcription is initiated. Enhancers potentiate promoter activity, temporally as well as spatially. Many promoters are known in the prior art that are transcriptionally active in a wide range of cell types.

As used herein, the term “operatively linked” refers to a linking between 2 polynucleotides in particular between an expression regulatory sequence (e.g. promoter) and a polynucleotide of interest.

As used herein, the term “vector” refers to an agent that is capable of transferring nucleic acid sequences to target cells (e.g., non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

As used herein, the term “host cell” may be any of a number commonly used cells in the production of exogenous polypeptides or proteins, including prokaryotic host cells.

As used herein the term “probiotic” is meant to designate live microorganisms which, they are integrated in a sufficient amount, exert a positive effect on health, comfort and wellness beyond traditional nutritional effects. Probiotic microorganisms have been defined as “Live microorganisms which when administered in adequate amounts confer a health benefit on the host” (FAO/WHO 2001).

As used herein, the term “transfection” refers to a wide variety of techniques commonly used for the introduction of exogenous DNA into a prokaryotic or eukaryotic host cell, e.g., electroporation, calcium-phosphate precipitation, DEAE-dextran transfection and the like. The host cell may be “transfected” with the vector of the invention by any conventional means known to the skilled artisan. For example transfection may be a transient transfection.

As used herein, the term “bile salt” has its general meaning in the art and are synthesized in the liver from cholesterol, conjugated with glycine or taurine and secreted in bile with cholesterol and lecithin. Exemplary bile salts include the salts of dihydroxy cholic acids, such as deoxycholic acid, glycodeoxycholic acid, taurodeoxycholic acid, chenodeoxycholic acid, glycochenodeoxycholic acid, and taurochenodeoxycholic acid, and trihydroxy cholic acids, such as cholic acid, glycocholic acid, and taurocholic acid. The alkaline salts include sodium, and potassium. In some embodiments, the bile salts are primary bile salts. Exemplary primary bile salts include the salts of dihydroxy cholic acids, such as chenodeoxycholic acid, glycochenodeoxycholic acid, and taurochenodeoxycholic acid, and trihydroxy cholic acids, such as cholic acid, glycocholic acid, and taurocholic acid.

As used herein, the term “output molecule” refers to a polynucleotide or polypeptide that is expressed in response to a particular signal, such as the presence of bile salts.

As used herein, the term “therapeutic polypeptide” refers to any kind of protein or polypeptide exerting a therapeutic action in a subject. The term “therapeutic polynucleotide” refers to any kind of polynucleotide exerting a therapeutic action in a subject.

As used herein, the term “subject” as used herein refers to any mammal organism. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the term “sample” refers to any volume of a liquid or suspension in which bile salts to be measured can be present in solution.

As used herein, the term “liver dysfunction” “or “hepatic dysfunction” refers to a state in which the liver function is decreased relative to a normal state. Hepatic dysfunction is characteristic of liver diseases.

As used herein, the term “non-alcoholic fatty liver disease” has its general meaning in the art and is intended to refer to the spectrum of disorders resulting from an accumulation of fat in liver cells in individuals with no history of excessive alcohol consumption. In the mildest form, NAFLD refers to hepatic steatosis. The term NAFLD is also intended to encompass the more severe and advanced form non-alcoholic steatohepatitis (NASH), cirrhosis, hepatocellular carcinoma, and virus- induced (e.g., HIV, hepatitis) fatty liver disease.

As used herein, the term “drug-induced liver disease” or “toxic liver injury” is used to describe those instances in which an active agent has caused injury to the liver.

As used herein, the term “alcoholic liver disease” or “alcoholic liver injury” refers to a disease caused by fat accumulation in liver cells caused at least in part by alcohol ingestion. Examples include, but are not limited to, diseases such as alcoholic simple fatty liver, alcoholic steatohepatitis (ASH), alcoholic hepatic fibrosis, alcoholic cirrhosis and the like. It should be noted that alcoholic steatohepatitis is also called alcoholic fatty hepatitis and includes alcoholic hepatic fibrosis.

As used herein, the term “risk” in the context of the present invention, relates to the probability that an event will occur over a specific time period and can mean a subject’s “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1- p) is the probability of no event) to no- conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

As used herein, the term “liver transplantation” has the common meaning in the art and includes partial and whole liver transplantation in which a liver of a donor is partially or wholly resected and partially or wholly transplanted into a recipient. Partial liver transplantation is classified by operation mode into orthotopic partial liver transplantation, heterotopic partial liver transplantation, and the like, and the present invention can be applied to any of them. In partial liver transplantation, a liver transplant or a partial liver transplant from a donor corresponding to about 30-50% of the normal liver volume of a recipient is typically transplanted as a graft into the recipient whose liver has been wholly resected.

As used herein, the term “transplant rejection” as used herein is defined as functional and structural deterioration of the organ due to an active immune response expressed by the recipient, and independent of non-immunologic causes of organ dysfunction. The transplant rejection may be acute or chronic. The term “acute rejection” as used herein refers to a rejection of the transplanted organ developing after the first 5-60 post-transplant days. It is generally a manifestation of cell-mediated immune injury. It is believed that both delayed hypersensitivity and cytotoxicity mechanisms are involved. The immune injury is directed against HLA, and possibly other cell-specific antigens expressed by the tubular epithelium and vascular endothelium. The term “chronic rejection” as used herein refers to a rejection of the transplanted organ developing after the first 30-120 post-transplant days. The term “chronic rejection” also refers to a consequence of combined immunological injury (e.g. chronic rejection) and non-immunological damage (e.g. hypertensive nephrosclerosis, or nephrotoxicity of immunosuppressants like cyclosporine A), taking place month or years after transplantation and ultimately leading to fibrosis and sclerosis of the allograft, associated with progressive loss of kidney function.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “effective amount” refers to a quantity sufficient of the prokaryotic host cell to achieve the beneficial effect.

As used herein, the term “biosensor device” has its general meaning in the art and refers to a device which converts an interaction between a sensor and a recognition molecule into a signal such as an electric signal, so as to measure or detect a target.

As used herein, term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). The DNA targeting endonuclease can be a naturally occurring endonuclease (e.g., a bacterial meganuclease) or it can be artificially generated (e.g., engineered meganucleases, TALENs, or ZFNs, among others). As used herein, the term “TALEN” has its general meaning in the art and refers to a transcription activator-like effector nuclease, an artificial nuclease which can be used to edit a target gene. As used herein, the term “ZFN” or “Zinc Finger Nuclease” has its general meaning in the art and refers to a zinc finger nuclease, an artificial nuclease which can be used to edit a target gene. As used herein, the term “CRISPR-associated endonuclease” has its general meaning in the art and refers to clustered regularly interspaced short palindromic repeats associated which are the segments of prokaryotic DNA containing short repetitions of base sequences.

As used herein, the term “food” refers to liquid (i.e. drink), solid or semi-solid dietetic compositions, especially total food compositions (food-replacement), which do not require additional nutrient intake or food supplement compositions. As used herein the term “food ingredient” or “feed ingredient” includes a formulation which is or can be added to functional foods or foodstuffs as a nutritional supplement. By “nutritional food” or “nutraceutical” or “functional” food, is meant a foodstuff which contains ingredients having beneficial effects for health or capable of improving physiological functions. By “food supplement”, is meant a foodstuff having the purpose of completing normal food diet.

### Polypeptides

The first object of the present invention relates to a bile salts sensing domain having an amino acid sequence as set forth in SEQ ID NO:3 wherein:
X₄₇ represents N, D, W, Y, T, V or F
X₄₈ represents Y or F
X₄₉ represents E, G, V, I, L, S or K
X₅₀ represents Q, V, H, A, T, D, L or S

SEQ ID NO:3 bile salt domain
YGQWYQHELAGITHDLRDLARLPGITIQKLSEQKLTFAIDQHQCSV-X₄₇-X₄₈-X₄₉-X_{5O}-KTLECTKN

In some embodiments, the bile salt domain comprises the amino acid sequence as set forth in SEQ ID: 25-33 as disclosed in Table A.

In some embodiments, the bile salt domain does not consist of the amino acid sequence as set forth in SEQ ID NO:24.

TABLE A

| | | | |
|---|---|---|---|
| Variant | Amino acid Seq | Nucleotide Seq | Bile salts sensing domain Amino acid seq |
| TcpPwt | NYEQ (SEQ ID NO:4) | aat tac gaa caa (SEQ ID NO:14) | YGQWYQHELAGITHDLRDLARLPGITIQKLSEQKLTFAI DQHQCSV-NYEQ-KTLECTKN (SEQ ID NO:24) |
| SV_3-3-3 | DFGV (SEQ ID NO:5) | gat ttt ggg gtg (SEQ ID NO:15) | YGQWYQHELAGITHDLRDLARLPGITIQKLSEQKLTFAI DQHQCSV- DFGV -KTLECTKN (SEQ ID NO:25) |
| SV_3-3-7 | WYVH (SEQ ID NO:6) | tgg tat gtt cat (SEQ ID NO:16) | YGQWYQHELAGITHDLRDLARLPGITIQKLSEQKLTFAI DQHQCSV- WYVH -KTLECTKN (SEQ ID NO:26) |
| SV_3-3-11 | YYIV (SEQ ID NO:7) | tat tat att gtt (SEQ ID NO:17) | YGQWYQHELAGITHDLRDLARLPGITIQKLSEQKLTFAI DQHQCSV- YYIV -KTLECTKN (SEQ ID NO:27) |
| SV_3-3-14 | TFLA (SEQ ID NO:8) | act ttt ctt gct (SEQ ID NO:18) | YGQWYQHELAGITHDLRDLARLPGITIQKLSEQKLTFAI DQHQCSV- TFLA -KTLECTKN (SEQ ID NO:28) |
| SV_3-3-16 | DFLT (SEQ ID NO:9) | gat ttt ctt act(SEQ ID NO:19) | YGQWYQHELAGITHDLRDLARLPGITIQKLSEQKLTFAI DQHQCSV- DFLT -KTLECTKN (SEQ ID NO:29) |
| SV_3-3-18 | VFSD (SEQ ID NO:10) | gtt ttt tcg gat (SEQ ID NO:20) | YGQWYQHELAGITHDLRDLARLPGITIQKLSEQKLTFAI DQHQCSV- VFSD -KTLECTKN (SEQ ID NO:30) |
| SV_3-3-19 | FFKA (SEQ ID NO:11) | ttt ttt aag gcg(SEQ ID NO:21) | YGQWYQHELAGITHDLRDLARLPGITIQKLSEQKLTFAI DQHQCSV- FFKA -KTLECTKN (SEQ ID NO:31) |
| SV_3-3-22 | YYVL (SEQ ID NO:12) | tat tat gtt ctt (SEQ ID NO:22) | YGQWYQHELAGITHDLRDLARLPGITIQKLSEQKLTFAI DQHQCSV- YYVL -KTLECTKN (SEQ ID NO:32) |
| SV_3-3-78 | FYES (SEQ ID NO:13) | ttt tat gag agt (SEQ ID NO:23) | YGQWYQHELAGITHDLRDLARLPGITIQKLSEQKLTFAI DQHQCSV- FYES -KTLECTKN (SEQ ID NO:33) |

A further object of the present invention relates to a TcpP polypeptide having a sequence as set forth in SEQ ID NO:34 wherein:
X₄₇ represents N, D, W, Y, T, V or F
X₄₈ represents Y or F
X₄₉ represents E, G, V, I, L, S or K
X₅₀ represents Q, V, H, A, T, D, L or S.

SEQ ID NO:34VVPYLVFSALYVALLPVIWWS-YGQWYQHELAGITHDL
RDLARLPGITIQKLSEQKLTFAIDQHQCSV-X₄₇-X₄₈-X₄₉-X₅₀-KTL
ECTKN

In some embodiments, the TcpP polypeptide comprises the amino acid sequence as set forth in SEQ ID: 36-44 as disclosed in Table B.

In some embodiments, the TcpP polypeptide does not consist of the amino acid sequence as set forth in SEQ ID: 35 as disclosed in Table B.

TABLE B

| | |
|---|---|
| Variant | TcpP polypeptide Amino acid seq |
| TcpPwt | VVPYLVFSALYVALLPVIWWS-YGQWYQHELAGITHDLRDLARLPGITIQKLSEQKLTFAIDQHQCSV-NYEQ-KTLECTKN (SEQ ID NO:35) |
| SV_3-3-3 | VVPYLVFSALYVALLPVIWWS-YGQWYQHELAGITHDLRDLARLPGITIQKLSEQKLTFAIDQHQCSV- DFGV -KTLECTKN (SEQ ID NO:36) |
| SV_3-3-7 | VVPYLVFSALYVALLPVIWWS-YGQWYQHELAGITHDLRDLARLPGITIQKLSEQKLTFAIDQHQCSV- WYVH -KTLECTKN (SEQ ID NO:37) |
| SV_3-3-11 | VVPYLVFSALYVALLPVIWWS-YGQWYQHELAGITHDLRDLARLPGITIQKLSEQKLTFAIDQHQCSV- YYIV -KTLECTKN (SEQ ID NO:38) |
| SV_3-3-14 | VVPYLVFSALYVALLPVIWWS-YGQWYQHELAGITHDLRDLARLPGITIQKLSEQKLTFAIDQHQCSV- TFLA -KTLECTKN (SEQ ID NO:39) |
| SV_3-3-16 | VVPYLVFSALYVALLPVIWWS-YGQWYQHELAGITHDLRDLARLPGITIQKLSEQKLTFAIDQHQCSV- DFLT -KTLECTKN (SEQ ID NO:40) |
| SV_3-3-18 | VVPYLVFSALYVALLPVIWWS-YGQWYQHELAGITHDLRDLARLPGITIQKLSEQKLTFAIDQHQCSV- VFSD -KTLECTKN (SEQ ID NO:41) |
| SV_3-3-19 | VVPYLVFSALYVALLPVIWWS-YGQWYQHELAGITHDLRDLARLPGITIQKLSEQKLTFAIDQHQCSV- FFKA -KTLECTKN (SEQ ID NO:42) |
| SV_3-3-22 | VVPYLVFSALYVALLPVIWWS-YGQWYQHELAGITHDLRDLARLPGITIQKLSEQKLTFAIDQHQCSV- YYVL -KTLECTKN (SEQ ID NO:43) |
| SV_3-3-78 | VVPYLVFSALYVALLPVIWWS-YGQWYQHELAGITHDLRDLARLPGITIQKLSEQKLTFAIDQHQCSV- FYES -KTLECTKN (SEQ ID NO:44) |

A further object of the present invention relates to a fusion protein wherein the TcpP polypeptide of the present invention is fused to a heterologous polypeptide.

In some embodiments, the heterologous polypeptide can be fused to the N-terminus or C-terminus of the TcpP polypeptide of the present invention.

In some embodiments, the TcpP polypeptide of the present invention is fused either directly or via a linker to the heterologous polypeptide.

In some embodiments, the heterologous polypeptide is a DNA binding domain.

In some embodiments, the heterologous polypeptide is a E coli CadC transcriptional activator DNA binding domain. In some embodiments, the E coli CadC transcriptional activator DNA binding domain comprises an amino acid sequence having at least 90% of identity with SEQ ID NO:45.

SEQ ID NO:45MQQPVVRVGEWLVTPSINQISRNGRQLTLEPRLIDLLV
FFAQHSGEVLSRDELIDNVWKRSIVTNHVVTQSISELRKSLKDNDEDSPV
YIATVPKRGYKLMVPVIWY

In some embodiments, the TcpP polypeptide of the present invention is fused to the E coli CadC transcriptional activator DNA binding domain via a linker.

In some embodiments, the linker consists of the amino acid sequence as set forth in SEQ ID NO:46.

SEQ ID NO:46SEEEGEEIMLSSPPPIPEAVPATDSPSHSLNIQNTATP
PEQSPVKSKR

In some embodiments, the fusion protein of the present invention comprises the amino acid sequence as set forth in SEQ ID NO:47 wherein:
X₄₇ represents N, D, W, Y, T, V or F
X₄₈ represents Y or F
X₄₉ represents E, G, V, I, L, S or K
X₅₀ represents Q, V, H, A, T, D, L or S.

SEQ ID NO:47MQQPVVRVGEWLVTPSINQISRNGRQLTLEPRLIDLLV
FFAQHSGEVLSRDELIDNVWKRSIVTNHVVTQSISELRKSLKDNDEDSPV
YIATVPKRGYKLMVPVIWY-SEEEGEEIMLSSPPPIPEAVPATDSPSHSL
NIQNTATPPEQSPVKSKR-VVPYLVFSALYVALLPVIWWS-YGQWYQHEL
AGITHDLRDLARLPGITIQKLSEQKLTFAIDQHQCSV-X₄₇-X₄₈-X₄₉-
X₅₀-KTLECTKN

In some embodiments, the fusion protein of the present invention comprises the amino acid sequence set forth in SEQ ID NO:48-57 as disclosed in Table C.

TABLE C

| | |
|---|---|
| Variant | Fusion protein Amino acid seq |
| TcpPwt | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV-NYEQ-KTLECTKN (SEQ ID NO:48) |
| SV_3-3-3 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- DFGV -KTLECTKN (SEQ ID NO:49) |
| SV_3-3-7 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR- |
| | VVPYLVFSALYVALLPVIWWS-YGQWYQHELAGITHDLRDLARLPGITIQKLSEQKLTFAIDQHQCSV- WYVH -KTLECTKN (SEQ ID NO:50) |
| SV_3-3-11 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- YYIV -KTLECTKN (SEQ ID NO:51) |
| SV_3-3-14 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- TFLA -KTLECTKN (SEQ ID NO:52) |
| SV_3-3-16 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- DFLT -KTLECTKN (SEQ ID NO:53) |
| SV_3-3-18 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- VFSD -KTLECTKN (SEQ ID NO:54) |
| SV_3-3-19 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- FFKA -KTLECTKN (SEQ ID NO:55) |
| SV_3-3-22 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- YYVL -KTLECTKN (SEQ ID NO:56) |
| SV_3-3-78 | MQQPVVRVGEWLVTPSINQISRNGRQLTLEPRLIDLLVFFAQHSGEVLSRDELIDNVWK RSIVTNHVVTQSISELRKSLKDNDEDSPVYIATVPKRGYKLMVPVIWY-SEEEGEEIMLSSPPPIPEAVPATDSPSHSLNIQNTATPPEQSPVKSKR-VVPYLVFSALYVALLPVIWWS-YGQWYQHELAGITHDLRDLARLPGITIQKLSEQKLTFAIDQHQCSV- FYES -KTLECTKN (SEQ ID NO:57) |

The polypeptides disclosed herein may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, California) and following the manufacturer’s instructions. Alternatively, the polypeptides and fusions proteins of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

### Polynucleotides

A further objet of the invention relates to a polynucleotide that encodes for a bile salt domain of the present invention.

A further object of the invention relates to a polynucleotide that encodes for a TcpP polypeptide of the present invention.

A further object of the invention relates to a polynucleotide that encodes for a fusion protein of the present invention.

A further object of the present invention relates to an expression cassette comprising the polynucleotide encoding for the fusion protein of the present invention and operably linked thereto control sequences allowing expression in a prokaryotic host cell.

Suitable expression control sequences include promoters that are applicable in the target host organism. Such promoters are well known to the person skilled in the art for diverse hosts from prokaryotic organisms and are described in the literature. For example, such promoters can be isolated from naturally occurring genes or can be synthetic or chimeric promoters. Likewise, the promoter can already be present in the target genome and will be linked to the polynucleotide by a suitable technique known in the art, such as for example homologous recombination.

In some embodiments, the promoter is selected from the group consisting of p14, p10, or p9 promoter having respectively a nucleic acid sequence as set forth in SEQ ID NO:58, SEQ ID NO:59 and SEQ ID NO:60.

SEQ ID NO:58>P14TTGACAATTAATCATCCGGCTCGTATAATGTGTG
GA

SEQ ID NO:59 >P10TTTCAATTTAATCATCCGGCTCGTATAATGTGT
GGA

SEQ ID NO:60 >p9TTGCCTCTTAATCATCGGCTCGTATAATGTGTGG
A

Expression cassettes according to the invention are particularly meant for an easy to use insertion into target polynucleotides such as vectors or genomic DNA. For this purpose, the expression cassette is preferably provided with nucleotide sequences at its 5′- and 3′-flanks facilitating its removal from and insertion into specific sequence positions like, for instance, restriction enzyme recognition sites or target sequences for homologous recombination as, e.g. catalyzed by recombinases.

### Vectors and Hosts Cells

A further object of the invention relates to vectors, particularly plasmids, cosmids, viruses and bacteriophages used conventionally in genetic engineering, that comprise a polynucleotide or an expression cassette of the present invention.

In some embodiments, the vectors of the present invention are suitable for the transformation of prokaryotic host cells. Methods which are well known to those skilled in the art can be used to construct recombinant vectors. In addition to the polynucleotide or expression cassette of the present invention, the vector may contain further genes such as marker genes which allow for the selection of said vector in a suitable host cell and under suitable conditions. Generally, the vector also contains one or more origins of replication. For genetic engineering, e.g. in prokaryotic host cells, the polynucleotides of the present invention or parts of these molecules can be introduced into plasmids. Expression vectors have been widely described in the literature. As a rule, they contain not only a selection marker gene and a replication origin ensuring replication in the host selected, but also a bacterial promoter and, in most cases, a termination signal for transcription. Between the promoter and the termination signal, there is in general at least one restriction site or a polylinker which enables the insertion of a coding nucleotide sequence. It is possible to use promoters ensuring constitutive expression of the gene and inducible promoters which permit a deliberate control of the expression of the gene. Bacterial promoter sequences possessing these properties are described in detail in the literature. Regulatory sequences for the expression in microorganisms (for instance E. coli) are sufficiently described in the literature. Inducible promoters are also possible. These promoters often lead to higher protein yields than do constitutive promoters.

A further object of the present invention relates to a method for producing a prokaryotic host cell capable of expressing the fusion protein of the invention comprising genetically engineering cells with an above-described polynucleotide, expression cassette or vector of the present invention.

A further object of the present invention relates to a prokaryotic host cell genetically engineered with an above-described polynucleotide, expression cassette or vector of the present invention, and to cells descended from such transformed cells and containing a polynucleotide, expression cassette or vector of the present invention and to cells obtainable by the above-mentioned method for producing the same.

In some embodiments, the prokaryotic host cell is selected among gram-positive or gram-negative bacteria.

In some embodiments, the prokaryotic host cell is selected among non-pathogenic bacteria. In some embodiments, the prokaryotic host cell is selected among bacteria that are derived from a normal internal ecosystem such as bacterial flora. In some embodiments, the prokaryotic host cell is selected among non-pathogenic bacteria that are derived from a normal internal ecosystem of the gastrointestinal tract. Non-limiting examples of nonpathogenic bacteria that are part of the normal flora in the gastrointestinal tract include bacteria from the genera Bacteroides, Clostridium, Fusobacterium, Eubacterium, Ruminococcus, Peptococcus, Peptostreptococcus, Bifidobacterium, Escherichia and Lactobacillus.

In some embodiments, the prokaryotic host cell is selected among anaerobic bacterial cells (e.g. cells that do not require oxygen for growth). Anaerobic bacterial cells include obligate anaerobic cells such as, for example, Bacteroides and Clostridium species. In humans, for example, anaerobic bacterial cells are most commonly found in the gastrointestinal tract.

In some embodiments, the prokaryotic host cell is elected from food grade bacteria. In some embodiments, the prokaryotic host cell is a probiotic.

In some embodiments, the prokaryotic host cell is E. Coli.

In some embodiments, the prokaryotic host cell is genetically engineered in such a way that it contains the introduced polynucleotide stably integrated into the genome. The transformation of the prokaryotic host cell with a polynucleotide or vector according to the invention can be carried out by standard methods. For example, calcium chloride transfection is commonly utilized for prokaryotic host cells. The prokaryotic host cell is cultured in nutrient media meeting the requirements of the particular prokaryotic host cell used, in particular in respect of the pH value, temperature, salt concentration, aeration, antibiotics, vitamins, trace elements etc.

In some embodiments, the prokaryotic host cell comprises a polynucleotide that encodes for the TcpH polypeptide having an amino acid sequence as set forth in SEQ ID NO:2. In some embodiments, said polynucleotide is operatively linked to the promoter p5 having the nucleic acid sequence as set forth in SEQ ID NO:61.

SEQ ID NO: 61>P5TTGACAATTAATCATCCGGCTCGTAATTTATGTG
GA

In some embodiments, the prokaryotic host cell of the present invention comprises at least one further polynucleotide encoding for an output molecule for which the expression is under the control of the fusion protein of the invention.

In particular, the binding of bile salts to the fusion protein triggers its oligomerization and thus allowing the oligomerization of the CadC transcriptional activator DNA binding domain which can then activate the expression of at least one further polynucleotide encoding for the output molecule that is placed under the control of CadBA promoter.

Accordingly, the prokaryotic host cell of the present invention further comprises a polynucleotide encoding for an output molecule operatively linked to a CadBA promoter. An exemplary nucleic acid for the CadBA promoter is represented by SEQ ID NO:62.

SEQ ID NO: 62>PCadBAATCCATTGTAAACATTAAATGTTTATCTTT
TCATGATATCAACTTGCGATCCTGATGTGTTAATAAAAAACCTCAAGTTC
TCACTTACAGAAACTTTTGTGTTATTTCACCTAATCTTTAGGATTAATCC
TTTTTTCGTGAGTAATCTTATCGCCAGTTTGG

In some embodiments, the output molecule is a polypeptide.

In some embodiments, the out molecule is a detection protein that can be detected by biological or physical means.

In some embodiments, the detection protein is a fluorescent protein. The advent of fluorescent proteins has allowed non-invasive intracellular labeling, which are easily detectable by optical means. The green fluorescent protein (GFP) from Aequorea Victoria is now the most widely used reporter gene in many organisms. Multiple variants with different spectral properties have been developed. In some embodiments, the prokaryotic host cell comprises different combinations of fluorescent proteins exhibiting energy transfer provide for differential fluorescence. In some embodiments, the detection protein is selected among luminescent proteins. Certain bacteria (e.g., Vibrio fischeri) have autoinducible luminescent genes that express luciferase, which causes cleaving of luciferin and emission of blue light. Bacteria produce signal molecules, N-acyl homoseine lactones (AELs) that enter bacterial cells and induce transcriptional activation of the genes LuxI, which encodes AHL synthetase, and LuxR, which encodes the AHL-dependent transcriptional activator. A sufficiently high concentration of AHL in the cell causes binding to the LuxR activator and transcription of the luminescence genes.

Alternatively, the detection proteins can be fusion proteins (e.g., green fluorescent protein-Fv) that have a detectable property and that are secreted from the cell. Thus, the secretion can be triggered by bile salts binding to the fusion protein of the present invention. In this case, the detection protein is produced in excess rather than in proportion to the bile salts binding.

In some embodiments, the detection can be performed using RNA aptamers specifically binding a fluorescent probe. Binding of the probe to the aptamer increases its fluorescence and allows detection of gene expression.

In some embodiments, the output molecule is a transcription factor that induces the expression of a detectable molecule or therapeutic molecule. In some embodiments, the output molecule is a repressor factor that represses the expression of a detectable molecule or therapeutic molecule.

In some embodiments, the output molecule is an endonuclease. In some embodiments, the transgene product of interest is an endonuclease that provides for site-specific knock-down of gene function, e.g., where the endonuclease knocks out an allele associated with a genetic disease. For example, where a dominant allele encodes a defective copy of a gene that, when wild-type, is a structural protein and/or provides for normal function, a site-specific endonuclease can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., the prokaryotic host cell of the present invention can be used to deliver both a site-specific endonuclease that knocks out a defective allele, and can be used to deliver a functional copy of the defective allele, resulting in repair of the defective allele, thereby providing for production of a functional protein. In some embodiments, the DNA targeting endonuclease of the present invention is a TALEN. In some embodiments, the DNA targeting endonuclease of the present invention is a ZFN. In some embodiments, the DNA targeting endonuclease of the present invention is a CRISPR-associated endonuclease. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd or the 4th nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector. In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” In some embodiments, the CRISPR-associated endonuclease is a Cpf1 nuclease.

In some embodiments, the output molecule is a therapeutic molecule, in particular a therapeutic polypeptide or a therapeutic polynucleotide.

Therapeutic polypeptides in the sense of the present invention are either proteins, which exist in nature, such as unmodified growth factors, or are designed therapeutic proteins, such as single-chain variable fragments of naturally occurring proteins or variants thereof. Therapeutic polypeptides exert their biological activity via different healing mechanisms. Therapeutic polypeptides are not only growth factors, but also other proteins with biological activity, such as but not limited to protease inhibitors or immune receptor antagonists. Therapeutic polypeptides used in the present innovation can be of form, in amino acid sequence and protein secondary and tertiary structure identical to naturally present, or may be modified or designed for improved action. For example, chimeric proteins can be formed by fusion of different therapeutic polypeptides. Therapeutic polypeptides are also bioactive molecules, not present in nature, such as single chain variable fragments, recombinant antibodies, peptides acting as antagonists, antibodies (e.g. neutralizing antibodies), nanobodies or soluble receptors. In some embodiments, the therapeutic polypeptide is a protein that binds tumor necrosis factor (TNF) or TNF receptors, a protein that binds integrins or integrin receptors, or fibroblast growth factor 19 (FGF19). Examples of proteins that bind TNF or TNF receptors include adalimumumab, certolizumab, golimumab, and infliximab and an anti-TNF Nanobody.

In some embodiments, the output molecule is a polynucleotide, in particular a therapeutic molecule. In some embodiments the output molecule is a ribonucleic acid (RNA). In some embodiments, the output molecule is an interfering RNA (RNAi).

Other kind of output signals include production of pigments via specific operons (like the violacein operon, or the expression of Flavin Mono Oxydase converting tryptophane into indigo), or by the expression of an enzyme which substrate exogenously supplied is transformed in a colorimetric product, like the enzyme Beta-galactosidase and its substrate X-gal for example.

More complex prokaryotic host cells with higher levels of functionality can be created using techniques developed in the field of cellular computation. In these methods, a cell serves as a biochemical computer, processing an input such as bile salts binding using internal logic gates to generate an output. Complex conditional responses to multiple inputs have been engineered for example by implementing AND, NOT, OR, XOR, and IMPLIES logic gates in E. coli cells. For instance, these gates can be implemented using DNA-binding proteins to regulate expression of recombinant vectors. Others systems can be used, such as, but not limited to, recombinase-based logic gates, nucleic acids-based logic gates, or protein-based logic gates. For more information on cellular computing, see R. Weiss, “Cellular Computation and Communications using Engineered Genetic Regulatory Networks,” Ph.D. Thesis, MIT, 2001; M. L. Simpson et al., “Whole-cell biocomputing,” Trends Biotechnol. 19: 317-323 (2001); Yaakov Benenson., « Biomolecular computing systems: principles, progress and potential ». «Nature Reviews Genetics, 13(7):455{468, 2012. ; Bonnet et al.,“Amplifying genetic logic gates” Science, 340(6132):599 {603, 2013. ; Brophy JAN and Voigt CA. « Principles of genetic circuit design ». Nature methods, 11(5):508{520, 2014., all of which are incorporated herein by reference.

### Diagnostic Methods

The prokaryotic host cell of the present invention constitutes whole-cells biosensor (“bactosensor”) that can be suitable for the detection and quantification of bile salts, in particular primary bile salts.

Accordingly a further object of the present invention relates to a method for detecting the presence of bile salts in a sample, comprising i) providing at least prokaryotic host cell of the present invention; b) contacting said prokaryotic host cell with the sample suspected of containing said bile salts for a time sufficient allowing the oligomerization of the fusion proteins binding and then the expression of the detection protein; and c) detecting the expression level of the detection protein wherein the expression level correlated with the amount of the bile salts present in the sample.

In some embodiments, the sample is bodily fluid sample. In some embodiments, the sample is selected from the group consisting of blood samples (including serum or plasma samples), urine samples, cerebrospinal samples, tear samples, saliva samples and synovial samples.

With the method of the present invention, it is possible to measure the concentration of bile salts solved in the sample over a molar range of several orders of magnitude.

The detection protein is assayed for and detected to quantify the bile salts, in particular primary bile salts. Typically, when the detection protein is a fluorescent protein, the fluorescence intensity on each cell can be read by methods known in the art such as flow cytometry, laser scanning cytometry, or imaging microscopy. In this way, the fluorescence intensity in all desired wavelength ranges on each individual cell can be detected. The amount or concentration of bile salts in the sample can then be determined using standard methods. In some embodiments, a calibration curve is constructed by measuring the detection protein expression (i.e., its fluorescence) when the cells are combined with samples containing known concentrations of bile salts. As long as a reproducible curve can be constructed, it is not necessary that the response be linear. The measured fluorescence intensity of the detection protein during an assay can then be correlated with the bile salts concentration in the sample using the calibration curve.

It will be appreciated by persons skilled in the art that the method of the present invention may be used in the detection, identification and quantification of bile salts in biological and non-biological samples, such as the diagnosis of disease in medicine or veterinarian science. These applications can be either commercial (in the sense of routine analyses) or serve pure research purposes. Because the method of the present invention may be employed using a virtually limitless variety of modalities, it enables the specific detection of thousands of different bile salts. To the degree that the whole-cell sensors are not destroyed, they may be reusable. In particular, the whole-cell sensors of the present invention are used as a medical diagnostics and disease management in the case of in vitro assays.

In particular, the whole-cell sensor of the present invention is particularly suitable for the diagnosis of a liver dysfunction in a subject.

Accordingly a further object of the present invention relates to a method for determining whether a subject has or is at risk of having a liver dysfunction comprising i) providing at least prokaryotic host cell of the present invention; b) contacting said prokaryotic host cell with a sample obtained from the subject for a time sufficient allowing the oligomerization of the fusion proteins binding and then the expression of the detection protein; and c) detecting the expression level of the detection protein wherein the expression level correlated with the amount of the bile salts present in the sample, and wherein said amount of the bile salts indicates whether the subject has or is at risk of having a liver dysfunction.

A number of acute or chronic pathological conditions lead to liver dysfunction. These include, but are not limited to liver abscess, liver cancer, either primary or metastatic, cirrhosis, such as cirrhosis caused by the alcohol consumption or primary biliary cirrhosis, amebic liver abscess, autoimmune hepatitis, biliary atresia, coccidioidomycosis disseminated, portal hypertension hepatic infections (such as hepatitis A virus, hepatitis B virus, hepatitis C virus, hepatitis D virus, or hepatitis E virus), hemochromatosis, hepatocellular carcinoma, pyogenic liver abscess, Reye’s syndrome, sclerosing cholangitis, Wilson’s disease, drug induced hepatotoxicity, or fulminant or acute liver failure. In some embodiments, the liver disease is a non-alcoholic fatty liver disease. In some embodiments, the liver disease is a drug-induced liver disease. In some embodiments, the liver disease is an alcoholic liver disease.

In some embodiments, the liver dysfunction may result from a viral infection. The liver is for instance involved in infections by hepatotropic viruses that replicate in the liver and for which the liver is the main target. These include hepatitis A, hepatitis B, hepatitis C, and hepatitis E viruses. In all of these infections, hepatitis and liver dysfunction arise as a consequence of the immune response and reparation mechanisms (e.g. fibrosis) to virus within the liver. In addition, the liver can be affected as part of a generalized host infection with viruses that primarily target other tissues, particularly the upper respiratory tract. Examples of viruses include the herpes viruses (Epstein-Barr virus, cytomegalovirus [CMV], and herpes simplex virus), parvovirus, adenovirus, and severe acute respiratory syndrome (SARS)-associated coronavirus (e.g. SARS-Cov-2).

In some embodiments, the method of diagnosing described herein is applied to a subject who presents symptoms of liver dysfunction without having undergone the routine screening to rule out all possible causes for liver dysfunction. The methods described herein can be part of the routine set of tests performed on a subject who presents symptoms of liver dysfunction such as jaundice, abdominal pain and swelling, swelling in the legs and ankles, itchy skin, dark urine color, pale stool color, bloody color stool, tar-colored stool, chronic fatigue, nausea or vomiting, loss of appetite, tendency to bruise easily... The method of the present invention can be carried out in addition of other diagnostic tools that include ultrasound evaluation (e.g. elastography), biopsy and/or quantification of at least one further biomarkers such as levels of blood AST, ALT, ALP, TTT, ZTT, total bilirubin, total protein, albumin, lactate dehydrogenase, choline esterase and the like.

In some embodiments, the subject underwent a liver transplantation. Accordingly, the present invention is particularly suitable for determining whether a liver transplant subject has or is at risk of having transplant rejection.

In some embodiments, the method of the present invention is particularly suitable for determining whether a subject suffering from a liver disease achieves a response to a therapy. The method is thus particularly suitable for discriminating responder from non-responder. A responder in the context of the present disclosure refers to a subject that will achieve a response, i.e. a subject who is under remission and more particularly a subject who does not suffer from liver dysfunction. A non-responder subject includes subjects for whom the disease does not show reduction or improvement after the treatment (e.g. the liver dysfunction remains stable or decreases). According to the present invention, the treatment consists in any method or drug that could be suitable for the treatment of liver dysfunction. Some liver problems can be treated with lifestyle modifications, such as stopping alcohol use or losing weight, typically as part of a medical program that includes careful monitoring of liver function. Each liver disease will have its own specific treatment regimen. For example, hepatitis A requires supportive care to maintain hydration while the body’s immune system fights and resolves the infection. Patients with gallstones may require surgery to remove the gallbladder. Other diseases may need long-term medical care to control and minimize the consequences of their disease. In patients with cirrhosis and end-stage liver disease, medications may be required to control the amount of protein absorbed in the diet. Other examples include operations required to treat portal hypertension.

The method of the present invention is particularly suitable for monitoring the efficiency of a therapy. Typically a decrease of binding capacity (e.g. between measures performed at different time intervals) indicates that subject does not achieve a response with the therapy. Conversely an increase of binding capacity (e.g. between measures performed at different time intervals) indicates that subject achieves a response with the therapy.

The method of the present is also particularly suitable for evaluating the effects of drugs under development in producing liver injury during a preclinical or clinical studies.

### Biosensors

The whole-cell sensor of the present invention could also be converted into a biosensor device that can be formed using the whole cell sensors of the present invention to be deployed in a microenvironment or microfluidic devices, or a collection of these devices in a multi-chip module or distributed wireless network. The biosensor device can respond to one or more specific chemical and/or physical inputs (e.g. heat or electrical current), generating outputs in the form of detection protein, and communicating with a physical transducer through calorimetric, electrochemical, or preferably fluorescence bioluminescence means. The 1 biosensor may thus comprise a detecting component that comprises the whole-sensor cell and a transducer component for converting a physical change or chemical change generated by the detecting component into an electric signal. According to the mechanism of biomarker detection, there are five types of transducers that may be used in biosensors of the present invention: optical (colorimetric, fluorescent, luminescent, and interferometric) transducers, mass-based (piezoelectric and acoustic wave) transducers, magnetic field based transducers, electrochemical (amperometric, potentiometric and conductometric) transducers, and calorimetric transducers. In some embodiments, the device may comprise additional measuring devices for measuring another parameter of interest. Typically the system comprises additional devices suitable for measuring a physiological phenotype. The physiological phenotype may include physiological parameters such as body temperature, pulse rate, blood pressure, respiratory rate, hydration status and the like. In some embodiments, the system includes an input/output module, an analysis module and a report generation module. The input/output module is configured to receive the amount of bile salts optionally in combinations with additional parameters optionally through the associated communication device. The analysis module is configured to analyze the different parameters that include the amount of bile salts. The report generation module is configured to generate the profile the subject on an analysis of the different parameters. In some embodiments, the system includes a sharing module to share one or more pre-formatted messages with one or more stakeholders based on comparison of the generated profile of the subject. In some embodiments, the system includes providing recommendations to the subject for alerting the subject about the risk of having a liver dysfunction. In some embodiments, the system includes enabling transmission of one more messages to an external operator (e.g. a physician) for alerting that the subject has or is at risk of having a liver dysfunction. Thus in some embodiments, the device comprises a communication device. Examples of communication devices include but may not be limited to mobile phones, tablets, desktop computers and the like. Various mediums can be used for connectivity including internet, intranet, Bluetooth, Wi-Fi and the like. In some embodiments the communication device is connected with a server. The measurements of one or more parameters measured by the measuring devices may be indeed transmitted wirelessly to a handheld device comprising a microprocessor. The handheld device may be a smartphone, a tablet device, a cell phone, a mobile internet device, a netbook, a notebook, a personal digital assistant, an internet phone, a holographic device, a holographic phone, a cable internet device, a satellite internet device, an internet television, a DSL internet device and a remote control.

### Kits

A further object of the present invention relates to a kit for performing the methods herein disclosed. The kit comprises one or more plurality of whole-cell sensor as above described and means for determining the expression level of the detection protein. Reagents for particular types of assays can also be provided in kits of the invention. In some embodiments, the kits comprise a device such as a biosensor as described above. In addition, the kits can include various diluents and buffers, labelled conjugates or other agents for the detection of the specifically immunocomplexes. Other components of a kit can easily be determined by one of skill in the art.

### Therapeutic Methods

The prokaryotic host cells are also particularly suitable for therapeutic purposes. In particular, the engineered prokaryotic host cells of the invention are suitable at operating in the gut and would be specifically activated upon arrival in the gut micro-environment in which bile salts are present. Said prokaryotic host cells are particularly suitable for expressing a therapeutic molecule (polynucleotide or polypeptide) in the gut. In presence of bile salts, in particular primary bile salts, the expression of the output therapeutic molecule can be triggered.

Thus a further object of the present invention relates to a method of therapy in a subject in need thereof comprising administering to the subject an effective amount of prokaryotic host cell of the invention that comprises a polynucleotide encoding for a therapeutic molecule.

Example of diseases that could be treated by the method of the present invention includes but are not limited to obesity, inflammatory bowel diseases, colorectal cancers, liver diseases and hepatobiliary diseases. In some embodiments, the disease or disorder is peptic ulcer disease, liver cirrhosis, inflammatory bowel disease, an infection, cancer, a vascular disorder, an adverse effect of a medication, or a blood clotting disorder. In some embodiments, the subject has or is at risk of having inflammatory bowel disease. Inflammatory bowel diseases (IBD) refer to a group of inflammatory conditions of the small intestine and colon. In some embodiments, the IBD is Crohn’s disease, ulcerative colitis, collagenous colitis, lymphocytic colitis, diversion colitis, Behçet’s disease, or indeterminate colitis.

In some embodiments, the prokaryotic host cell is administered into the gut.

In some embodiments, the prokaryotic host cell of the present invention is encapsulated in order to be protected against the stomach. Accordingly, in some embodiments the prokaryotic host cell of the present invention is formulated in compositions in an encapsulated form so as significantly to improve their survival time. In such a case, the presence of a capsule may in particular delay or prevent the degradation of the prokaryotic host cell in the gastrointestinal tract. It will be appreciated that the compositions of the present embodiments can be encapsulated into an enterically-coated, time-released capsule or tablet. The enteric coating allows the capsule/tablet to remain intact (i.e., undissolved) as it passes through the gastrointestinal tract, until such time as it reaches the intestine. Methods of encapsulating live bacterial cells are well known in the art (see, e.g., U.S. Pat. to General Mills Inc. such as U.S. Pat. No. 6,723,358). For instance, encapsulation can be done with enteric coatings that are preferably methacrylic acid- alkyl acrylate copolymers, such as Eudragit® polymers. Poly(meth)acrylates have proven particularly suitable as coating materials.

In some embodiments, the prokaryotic host cell of the present invention is administered to the subject in the form of a food composition. In some embodiments, the food composition is selected from complete food compositions, food supplements, nutraceutical compositions, and the like. The composition of the present invention may be used as a food ingredient and/or feed ingredient. The food ingredient may be in the form of a solution or as a solid—depending on the use and/or the mode of application and/or the mode of administration. Food and food supplement compositions are for example fermented dairy products or dairy-based products, which are preferably administered or ingested orally one or more times daily. Fermented dairy products can be made directly using the bacteria according to the invention in the production process, e.g. by addition to the food base, using methods known per se. In such methods, the strain(s) of the invention may be used in addition to the micro-organism usually used, and/or may replace one or more or part of the micro-organism usually used. Fermented dairy products include milk-based products, such as (but not limited to) deserts, yoghurt, yoghurt drinks, quark, kefir, fermented milk-based drinks, buttermilk, cheeses, dressings, low fat spreads, fresh cheese, soy-based drinks, ice cream, etc. Alternatively, food and/or food supplement compositions may be non-dairy or dairy non fermented products (e.g. strains or cell-free medium in non-fermented milk or in another food medium). Non-fermented dairy products may include ice cream, nutritional bars and dressings, and the like. Non-dairy products may include powdered beverages and nutritional bars, and the like.

In some embodiments, the food composition that comprises the prokaryotic host cell of the present invention contains at least one prebiotic i.e. a food substance intended to promote the growth of the prokaryotic host cell of the present invention in the intestines. The prebiotic may be selected from the group consisting of oligosaccharides and optionally contains fructose, galactose, mannose, soy and/or inulin; and/or dietary fibers.

In the context of the present invention, the amount of the prokaryotic host cell administered to the subject will depend on the characteristics of the individual, such as general health, age, sex, body weight... The skilled artisan will be able to determine appropriate dosages depending on these and other factors. For example, the prokaryotic host cell shall be able to generate a colony is sufficient to generate a beneficial effect on the subject. If the prokaryotic host cell is administered in the form of a food product, it typically may comprise between 10³ and 10¹² cfu of the prokaryotic host cell of the present invention per g of the dry weight of the food composition.

### Screening Methods

The prokaryotic host cell of the present invention is also particularly suitable for screening purposes. In particular, the prokaryotic host cell of the present invention is particularly suitable for screening of drugs that are suitable e.g. for inhibiting pathogen signaling pathways. In this case, the system is a rewired version of Vibrio cholerae virulence activation pathway. As it is rebuilt in a non-pathogenic prokaryotic host cell, such as E.coli, and that activation can be monitored easily with the detection of a detectable output molecule, this system could serve as a platform for high-throughput screening of compound libraries to identify new inhibitors (or activators) of V. cholerae virulence that could be used as therapeutics.

Thus a further object of the present invention relates to a method of screening a plurality of test substances comprising i) contacting a population of prokaryotic host cells of the present invention with said plurality of test substances in presence of an amount of bile salts, and ii) selecting the test substances capable of modulating the expression of the output molecule.

The test substance of the invention may be selected from a library of substances previously synthesised, or a library of substances for which the structure is determined in a database, or from a library of substances that have been synthesised de novo. The test substance may be selected from the group of (a) proteins or peptides, (b) nucleic acids and (c) organic or chemical substances.

In some embodiments, the method comprises the steps consisting of comparing the expression level of the output molecule (e.g. detection protein) with the expression level determined in the absence of the test substance and positively selecting the test substance that provides a decrease or an increase in the expression level of the output molecule.

The invention will be further illustrated by the following figures and examples. However, these examples and figures should not be interpreted in any way as limiting the scope of the present invention.

### FIGURES

FIG. 1. Rewiring virulence sensing into modularized synthetic receptor platform. Schematic diagram of isolation and rewiring the bile salt sensing module - transmembrane and periplasmic domain of TcpP and TcpH from pathogen V.cholerae into modularized E. coli synthetic receptor platform. The sensing domain TcpP detecting bile salts is plugged into a synthetic receptor which activates GFP expression when bile salts are present.

FIG. 2. System performance of constitutively expressed synthetic CadC-TcpP_TcpH receptor platform. (A) architecture of the expression system. encoding CadC-TcpP and TcpH controlled by constitutive promoters (P14, P10, P9 for TcpP and P5 for TcpH). (B) response of the different construcs to increasing concentration of bile salts (taurocholic acid). The area labeled with symbols (Δ) indicates the serum bile salt concentration for healthy people (4.8 ± 0.6 µM,Δ), patients with chronic liver disease (51.3 ± 3.8 µM, ΔΔ), and patients with drug induced liver injuries (110 ± 3.4 µM, ΔΔΔ).(C) The activation fold of P14, P10 and P9 controlled CadC-TcpP are measured under the presence of 120 µM Taurocholic acid.

FIG. 3. Specificity testing of the rewired TcpP-Cadc System across different bile salts.

FIG. 4. Sensitivity engineering of TcpP bile salt sensing domain Taurocholic acid titration curve of selected TcpP functional variants.

FIGS. 5. Response of bile salt bactosensor in clinical samples. (A) Response of bacterial cells harboring synthetic P9-CadC-TcpP_P5-TcpH receptor in clinical serum samples. (B) Response of bacterial cells harboring synthetic P9-CadC-TcpP_P5-TcpH receptor in clinical urine samples.

FIG. 6. Colorimetric assay for bactosensor mediated bile salts detection. Bile salt specificity profile of the TcpP SV_3-3-18 variant characterized using SV_3-3-18-LacZ sensor. Response of SV_3-3-18-LacZ was quantified as ΔA580 (the difference in absorbance at 580 nm (A580) with or without ligand bile salts). The bar graph and error corresponds to the mean value of three experiments performed in triplicate. Cells growing in exponential phase were incubated with bile salts for 4 hours before flow cytometry analysis.

FIG. 7. Bactosensor-based pathological bile salt detection in clinical samples. Comparing the analysis result of 21 clinical serum samples between TcpP18LacZ and a bile salt assay kit. The response of TcpP SV_3-3-18-LacZ is shown in black bars, left axis. The serum total bile salt concentration measured by a bile salt enzymatic assay kit is labeled in green asterisks, right axis.

### EXAMPLE 1: TCPP SENSOR

The liver is a vital organ coordinating metabolic, detoxification, and immunological processes. Liver diseases including hepatitis, cirrhosis, fatty liver disease and cancer are major public health problems and require large-scale screening methods for prevention, diagnosis, and therapeutic monitoring. Liver function is usually monitored by quantifying serum enzymatic activities and bilirubin, but these markers are detectable when damage has already progressed, and are not entirely specific. Liver function is usually monitored by quantifying several enzymatic activities simultaneously due to their lack of specificity. Serum and urinary bile salts are alternative biomarkers for early diagnostics of liver dysfunction, yet their current detection methods are impractical and hard to scale.

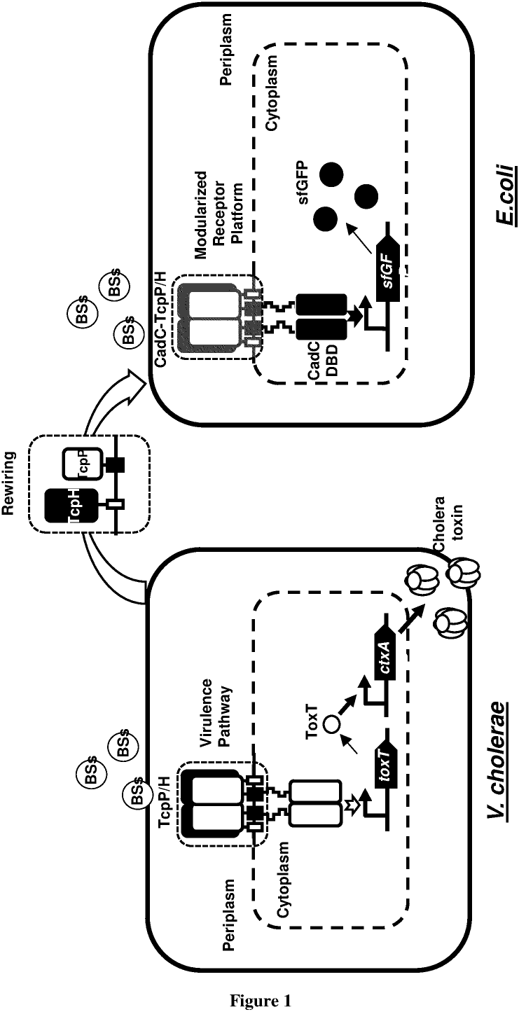

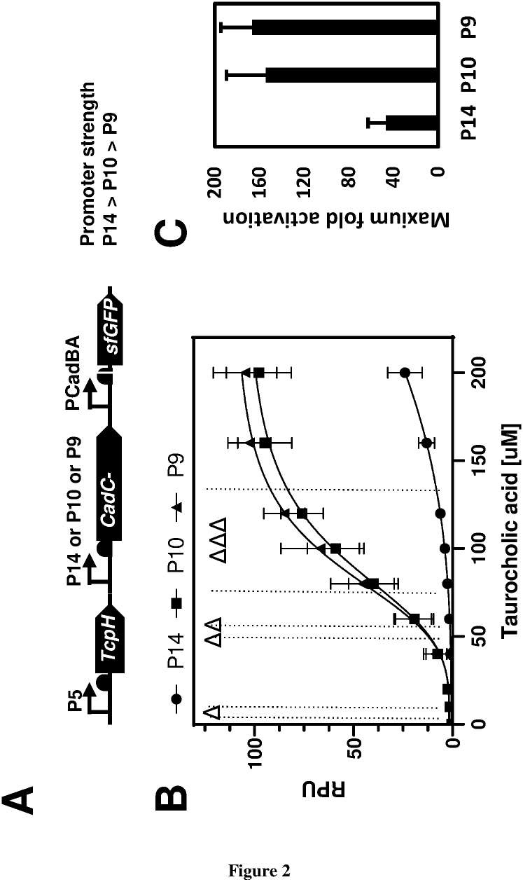

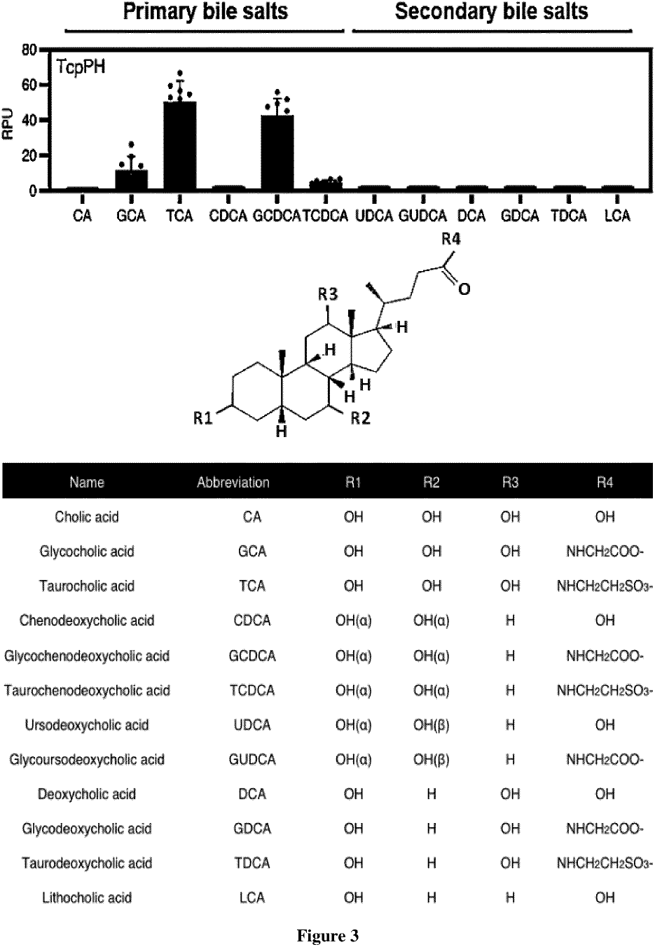

Here we engineered a bacterial biosensor based on non-pathogenic E. coli detecting pathological concentrations of bile salts in clinical samples. We repurposed the bacterial one-component TcpP bile salts sensing domain from Vibrio cholerae which controls activation of virulence operons when the pathogen enters the gut. We engineered synthetic bile salt receptors using TcpP as sensing domains connected to E. coli CadC system which activates transcription upon dimerization (FIG. 1). The performance of the system was assayed for various selection of promoters (FIGS. 2A, B) and we can show that fine tunable response may be reach by changing expression levels of the bile salt receptor. We also test the system across different bile salts (FIG. 3). We measured the response of the bactosensor to a panel of twelve different bile salts, including both primary and secondary types. Interestingly, while no sensing module was specific for a single bile salt species, the CadC-TcpP system was highly specific for primary conjugated bile salts and did not respond to secondary bile salts.

We aimed to identify key residues determining the sensitivity of the TcpP sensing module, and targeted those to improve synthetic receptor sensitivity and LOD. To do so, we coupled comprehensive mutagenesis with functional screening and Next-Generation Sequencing (NGS), an approach which supports the identification of functional variants together with the sequence determinants within the local structural motifs. Transition from intramolecular to intermolecular disulfide bonds between TcpP monomers is a key determinant of TcpP response to bile salts and is mediated by two cysteine residues, Cys207 and Cys218. By performing multiple sequence alignments of different TcpP bacterial homologs (data not shown), we found a significant conservation of the amino-acids flanked by these two cysteines (data not shown). Secondary structure prediction and ab initio 3D prediction using the Rosetta modelling suite (data not shown) suggested that each cysteine was located in rigid beta-sheets separated by a flexible loop region between Asn211 and Gln214. This loop propensity to form a turn would allow the two beta sheets and the cysteines to come in close proximity and form an intramolecular disulfide bond. We hypothesized that the flexibility of the turn region between Cys207 and Cys218 was a key parameter controlling the transition rate between the two states, and that altering its amino-acid composition could change the system’s sensitivity to bile salts.

We thus built a comprehensive mutational library (NNK x 4, theoretical library complexity ≅ 1.05 × 10⁶ variants) targeting the NYEK residues inside the turn, and cloned it into the plasmid constitutively expressing CadC-TcpP and producing GFP in response to bile salts (data not shown). The resulting library was induced with TCA, and GFP-positive variants were isolated by fluorescence-activated cell sorting (FACS). We performed three rounds of enrichment (200 µM of TCA as ligand in 1st and 2nd rounds of selection, and 20 µM for the 3rd round) and observed an increasing fraction of the cell population responding to different ligand concentrations (20 to 80 µM) (data not shown). We collected, cultured, and sequenced single variants and tested their response to TCA (data not shown). We found that comprehensive mutagenesis of residues Asn210 to Gln213 could alter the limit of detection, the sensitivity, and the fold activation of our biosensor. The 3.3-fold difference in LOD between SV_3-3-18 and SV_3-3-22 (EC50 from 28.3 to 92.5 µM) indicated the broad range of sensitivity engineering obtained by mutating the loop region of the TcpP sensing module. Further kinetic analysis revealed that the sequence variation of this loop region changes reaction speed and system interaction of bile salts with the synthetic receptor, and variant SV_3-3-18 has 13-fold increase in ligand affinity and faster response at low ligand concentration compared to wt TcpP (data not shown).

To better understand the sequence features influencing the response of TcpP to bile salts, we sequenced the whole pool of enriched variants by next-generation sequencing (NGS). Surprisingly, the sequence features of functional variants were different from those expected from natural TcpP homologs (data not shown). First, and in contrast with wt TcpP homologs, we observed a strong depletion of long-chain, negatively charged amino-acids (Asp and Glu) along with long-chain polar amino-acids (Asn and Gln) at position 211. Lysine at position 211 also appeared to be depleted in functional variants (despite being commonly found at this position in other TcpP homologous proteins). Second, amino acids with bulky aromatic side chain such as Phe and Tyr, and hydrophobic side chain such as Leuwere highly conserved in selected functional variants, strongly indicating the important role of hydrophobic residues at position 211 in the C-terminal loop region for the function of V. cholerae TcpP. The best engineered variant was SV_3-3-18.

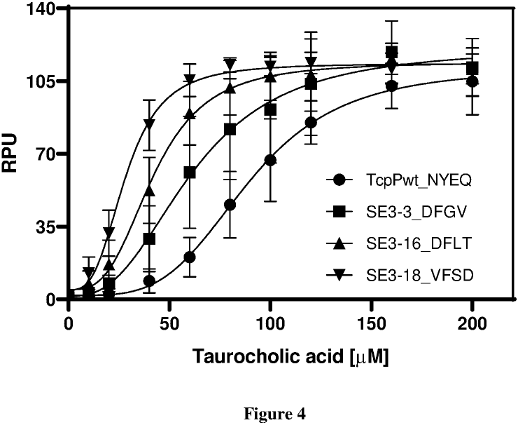

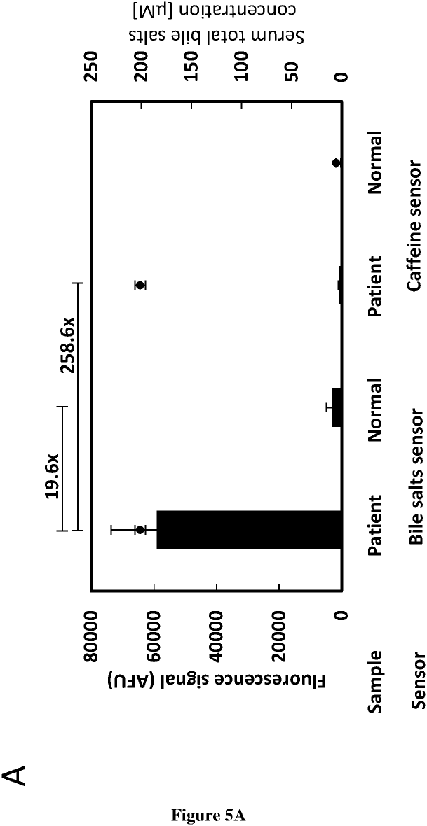

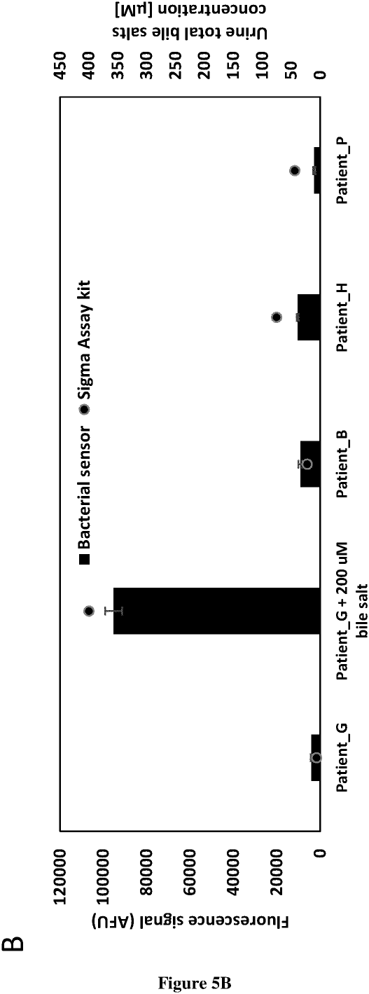

By performing multiple rounds of directed evolution of the TcpP sensor we obtained a collection of variants with a lower limit of detection and a higher sensitivity (Table 1 and FIG. 4). Finally, we show that our bactosensor can detect pathological bile-salt concentrations in samples from patients with liver dysfunction (FIGS. 5A, B).

TABLE 1

list of different variants and their characteristics

| # Variant | Sequence | EC50 (µM) | Response EC50 (RPU) | Max fold change |
|---|---|---|---|---|
| SV_3-3-22 | YYVL (SEQ ID NO: 12) | 92.531 | 56.19 | 69.06 |
| TcpPwt | NYEQ (SEQ ID NO: 4) | 89.405 | 56.5 | 164.74 |
| SV_3-3-7 | WYVH (SEQ ID NO: 6) | 86.029 | 53.37 | 51.31 |
| SV_3-1-78 | FYES (SEQ ID NO: 13) | 84.668 | 59.62 | 69.06 |
| SV_3-3-11 | YYIV (SEQ ID NO: 7) | 82.167 | 53.83 | 31.76 |
| SV_3-3-14 | TFLA (SEQ ID NO: 8) | 81.636 | 62.97 | 222.67 |
| SV_3-3-3 | DFGV (SEQ ID NO: 5) | 61.48 | 61.18 | 142.21 |
| SV_3-3-19 | FFKA (SEQ ID NO: 11) | 59.186 | 61.29 | 127.75 |
| SV_3-3-16 | DFLT (SEQ ID NO: 9) | 42.058 | 59.34 | 75.73 |
| SV_3-3-18 | VFSD (SEQ ID NO: 10) | 28.344 | 58.99 | 84.92 |

Our work paves the way to a sensitive, scalable, and affordable screening platform for liver dysfunction, that could be deployed in point-of-care or at-home settings and enable large scale monitoring of liver associated diseases. This work also shows how synthetic biology can help address global healthcare challenges while providing tools to decipher and target basic cellular mechanisms, in this case pathogens signaling.

### EXAMPLE 2: COLORIMETRIC ASSAY

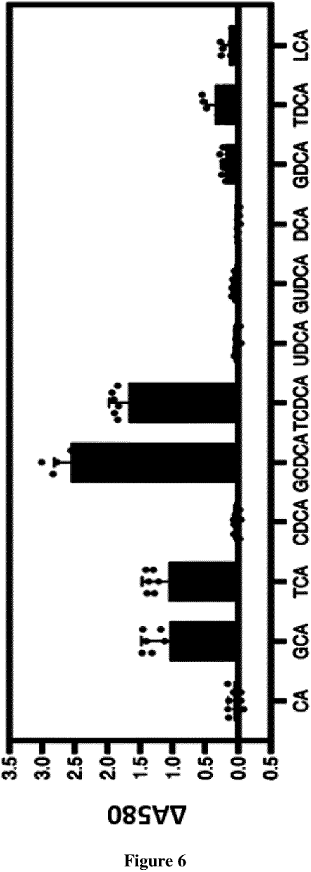

Colorimetric assay provides a simple and intuitive method for simple and direct estimation of test results by the naked eye. In addition, colorimetric assays support straightforward development of quantitative assays using smartphone-based platforms for POC or home-based diagnosis. We used SV_3-3-18 variant coupled with the reporter beta-galactosidase LacZ (termed SV_3-3-18-LacZ) and its substrate chlorophenol red-β-D-galactopyranoside (CPRG) to provide a colorimetric output (data not shown). Similarly to the biosensor equipped with a GFP output, the bile salt specificity profile of the SV_3-3-18-LacZ system was slightly shifted from TCA to GCDCA (FIG. 6). We thus evaluated the LOD and signal output threshold of SV_3-3-18-LacZ in response to increasing concentrations of GCDCA. We also explored the influence of varying cell density and incubation time (data not shown). Increasing cell density or incubation time both improved the dynamic and operating ranges of SV_3-3-18-LacZ; however, background signal also increased. After optimization, the SV_3-3-18-LacZ demonstrated linear response to GCDCA within the concentration from 0 to 40 µM in one hour (data not shown). In addition, adjusting cell density and incubation times allows its threshold activation level to match various clinical levels associated with specific liver-related medical conditions.

### EXAMPLE 3: BACTOSENSOR-MEDIATED DETECTION OF ELEVATED BILE SALTS LEVELS IN SERUM FROM PATIENTS WITH LIVER TRANSPLANT

We tested the sensor on samples from patients having undergone liver transplantation. After liver transplant, the main complications are bile ducts stenosis and acute cellular rejection.

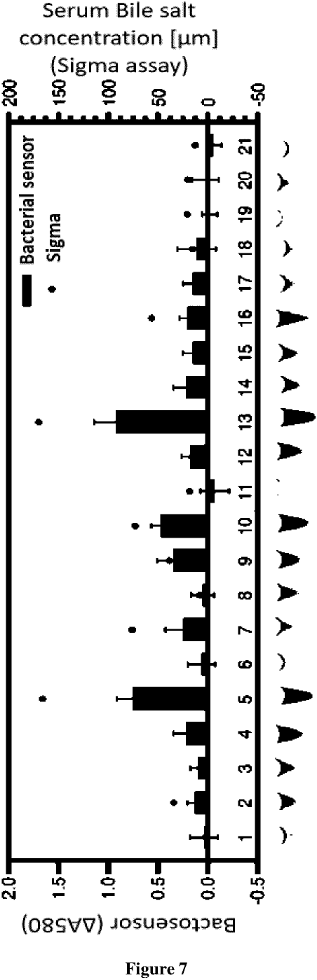

We tested our bactosensor in clinical 21 serum samples from liver transplantation patients (data not shown). The patients were followed at the Montpellier hospital after their liver transplant, most of them having been performed in the last 2 years. These patients had received a liver transplant for end-stage liver disease as a result of alcoholic related liver disease or non-alcoholic fatty liver disease, chronic cholangitis or liver cancer. A complete hepatic check-up was performed, and serum bile salts were also measured using an enzymatic assay (data not shown). We found that patients who had a high potential of acute cellular rejection (ACR) after liver transplantation (serum bile acid > 37 µM) had significant and visible colorimetric signal changes (data not shown) in bactosensor assays. Three patients with elevated serum bile salts concentration raised our attention. Two of them presented abnormalities in their hepatic enzymatic values (ASAT, ALAT, GGT, Pal, and bilirubin). For these patients, the bile salt bactosensor produced the strongest colorimetric change easily detectable with the naked eye (FIG. 7). These results indicate that our bactosensor is able to provide a simple, reliable, and cost-effective method for monitoring patient condition after liver transplantation.

### REFERENCES

Throughout this application, various references describe the state of the art to which this invention pertains. The disclosures of these references are hereby incorporated by reference into the present disclosure.

1. A bile salts sensing domain having an amino acid sequence as set forth in SEQ ID NO:3 wherein:
X₄₇ represents N, D, W, Y, T, V or F
X₄₈ represents Y or F
X₄₉ represents E, G, V, I, L, S or K
X₅₀ represents Q, V, H, A, T, D, L or S
and provided that the bile salt domain does not consist of the amino acid sequence as set forth in SEQ ID NO:24.

2. The bile salts sensing domain of claim 1 that comprises the amino acid sequence as set forth in at least one of SEQ ID NOS: 25-33.

3. A toxin coregulated pilus biosynthesis protein P of Vibrio cholerae (TcpP) polypeptide having a sequence as set forth in SEQ ID NO:34 wherein:
X₄₇ represents N, D, W, Y, T, V or F
X₄₈ represents Y or F
X₄₉ represents E, G, V, I, L, S or K
X₅₀ represents Q, V, H, A, T, D, L or S
and provided that the TcpP polypeptide does not consist of the amino acid sequence as set forth in SEQ ID: 35.

4. The TcpP polypeptide of claim 3 that comprises the amino acid sequence as set forth in at least one of SEQ ID NOS: 36-44.

5. A fusion protein wherein a TcpP polypeptide is fused to a heterologous polypeptide, and wherein the TcpP polypeptide has a sequence as set forth in SEQ ID NO:34 wherein:
X₄₇ represents N, D, W, Y, T, V or F
X₄₈ represents Y or F
X₄₉ represents E, G, V, I, L, S or K
X₅₀ represents Q, V, H, A, T, D, L or S.

6. The fusion protein of claim 5 wherein the heterologous polypeptide is a DNA binding domain.

7. The fusion protein of claim 5 wherein the heterologous polypeptide is a an E coli CadC transcriptional activator DNA binding domain .

8. The fusion protein of claim 5 wherein the TcpP polypeptide is fused either directly or via a linker to the heterologous polypeptide.

9. The fusion protein of claim 8 wherein the linker consists of the amino acid sequence as set forth in SEQ ID NO:46.

10. The fusion protein of claim 8 that comprises the amino acid sequence as set forth in SEQ ID NO:47 wherein:
X₄₇ represents N, D, W, Y, T, V or F
X₄₈ represents Y or F
X₄₉ represents E, G, V, I, L, S or K
X₅₀ represents Q, V, H, A, T, D, L or S.

11. The fusion protein of claim 10 that comprises the amino acid sequence set forth in one of SEQ ID NOS:48-57.

12. A polynucleotide that encodes the fusion protein of claim 5.

13. An expression cassette comprising the polynucleotide of claim 5 operably linked to control sequences allowing expression in a prokaryotic host cell.

14. The expression cassette of claim 13 wherein the promoter is selected from the group consisting of p14, p10, and p9 promoter having respectively a nucleic acid sequence as set forth in SEQ ID NO:58, SEQ ID NO:59 and SEQ ID NO:60.

15. A prokaryotic host cell genetically engineered with the polynucleotide of claim 12 or the an expression cassette comprising the polynucleotide.

16. The prokaryotic host cell of claim 15 that is selected from a bacterium from the genera Bacteroides, Clostridium, Fusobacterium, Eubacterium, Ruminococcus, Peptococcus, Peptostreptococcus, Bifidobacterium, Escherichia and Lactobacillus.

17. The prokaryotic host cell of claim 15 that is an Escherichia coli bacterium.

18. The prokaryotic host cell of claim 15 that comprises a polynucleotide that encodes for the TcpH polypeptide having an amino acid sequence as set forth in SEQ ID NO:2, wherein optionally said polynucleotide is operatively linked to the promoter p5 having the nucleic acid sequence as set forth in SEQ ID NO:61.

19. The prokaryotic host cell of claim 15 that comprises at least one further polynucleotide encoding for an output molecule for which the expression is under the control of the fusion protein in which a TcpP polypeptide is fused to a polypeptide, and wherein the TcpP polypeptide has a sequence as set forth in SEQ ID NO:34 wherein:
X₄₇ represents N, D, W, Y, T, V or F
X₄₈ represents Y or F
X₄₉ represents E, G, V, I, L, S or K
X₅₀ represents Q, V, H, A, T, D, L or S.

20. The prokaryotic host cell of claim 15 that further comprises a polynucleotide encoding for an output molecule operatively linked to the CadBA promoter of SEQ ID NO:62.

21. The prokaryotic host cell of claim 20, wherein the output molecule is a detection protein.

22. The prokaryotic host cell of claim 20, wherein the output molecule is a therapeutic polypeptide.

23. A method for detecting the presence of bile salts in a sample suspected of containing said bile salts, comprising i) providing at least one prokaryotic host cell of claim 21; b) contacting said at least one prokaryotic host cell with the sample suspected of containing said bile salts for a time sufficient to allow the oligomerization of the fusion proteins encoded by the at least one prokaryotic host cell to bind and then to express the detection protein; and c) detecting the expression level of the detection protein, wherein the expression level correlate with the amount of the bile salts present in the sample.

24. A method for determining whether a subject has or is at risk of having a liver dysfunction and treating the subject comprising i) providing at one least prokaryotic host cell of the claim 21; b) contacting said at one least prokaryotic host cell with a sample obtained from the subject for a time sufficient to allow the oligomerization of the fusion proteins encoded by the at least one prokaryotic host cell to bind and then to express the detection protein; c) detecting the expression level of the detection protein, wherein the expression level correlateds with the amount of the bile salts present in the sample, and d) treating the subject determined to have an elevated amount of bile salts with a liver dysfunction treatment.

25. A method of treating obesity, inflammatory bowel disease, colorectal cancer, liver disease or hepatobiliary disease in a subject in need thereof comprising administering to the subject an effective amount of the prokaryotic host cell of claim 22.

26. (canceled)

27. A method of screening a plurality of test substances comprising i) contacting a population of prokaryotic host cells of claim 21 with said plurality of test substances in the presence of an amount of bile salts, and ii) selecting the test substances capable of modulating the expression of the output molecule.

28. The fusion protein of claim 7 wherein the E coli CadC transcriptional activator DNA binding domain comprises an amino acid sequence having at least 90% of identity with SEQ ID NO:45.

29. The prokaryotic host cell of claim 21, wherein the detection protein is a fluorescent protein.
